(19) United States
(12) Patent Application Publication
(10) Pub. No.: US 2013/0253440 A1
(43) Pub. Date: Sep. 26, 2013

(54) Drug Delivery Device

(71) Applicants: Christopher James Smith, Cheshire (GB); Carmen Patricia Keating, Nowendoc (AU); David Martin Leak, Lake Hopatcong, NJ (US); Malcolm Stanley Boyd, Warwickshire (GB)

(72) Inventors: Christopher James Smith, Cheshire (GB); Carmen Patricia Keating, Nowendoc (AU); David Martin Leak, Lake Hopatcong, NJ (US); Malcolm Stanley Boyd, Warwickshire (GB)

(73) Assignee: SANOFI-AVENTIS DEUTSCHLAND GMBH, Frankfurt am Main (DE)

(21) Appl. No.: 13/989,355

(22) PCT Filed: Nov. 28, 2011
(86) PCT No.: PCT/EP2011/071147

§ 371 Date: May 23, 2013

Related U.S. Application Data
(60) Provisional application No. 61/433,822, filed on Jan. 18, 2011.

(30) Foreign Application Priority Data
Nov. 29, 2010 (EP) 10192845.5

(51) Int. Cl. A61M 5/315

(52) U.S. Cl. CPC A61M 5/31545
USPC 604/246

(57) ABSTRACT

A drug delivery device having a dose limiting system. The drug delivery device includes a first dose setting mechanism operably coupled to a primary reservoir holding a first medicament. The first dose setting mechanism includes a first dose setter and is a variable dose setting mechanism. The device further includes a second dose setting mechanism operably coupled to a secondary reservoir holding a second medicament, and the second dose setting mechanism includes a second dose setter. Still further, the device includes a dose limiting system. The dose limiting system operably couples the variable dose setting mechanism and the fixed dose setting mechanism. Further, the dose limiting system is configured to limit a settable amount of a dose of the second medicament a user can set using the second dose setter based on an amount of a variable dose that a user sets using the first dose setter.

### CROSS REFERENCE TO RELATED APPLICATIONS

The present application is a U.S. National Phase application pursuant to 35 U.S.C. §371 of International Application No. PCT/EP2011/071147 filed Nov. 28, 2011, which claims priority to European Patent Application No. 10192845.5 filed Nov. 29, 2010 and U.S. Provisional Patent Application No. 61/433,822 filed Jan. 18, 2011. The entire disclosure contents of these applications are herewith incorporated by reference into the present application.

### FIELD OF INVENTION

This present patent application relates to medical devices and methods of delivering at least two drug agents from separate reservoirs using devices having only a single dispense interface. The drug agents are contained in two or more multiple dose reservoirs, containers or packages, each containing independent (single drug compound) or pre-mixed (co-formulated multiple drug compounds) drug agents. The disclosed method and system is of particular benefit where the therapeutic response can be optimized for a specific target patient group, through control and definition of the therapeutic profile.

### BACKGROUND

Certain disease states require treatment using one or more different medicaments. Some drug compounds need to be delivered in a specific relationship with each other in order to deliver the optimum therapeutic dose. The disclosed method and system is of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two active medicaments or “agents” simultaneously. The two active agents may interact with each other during the long-term, shelf life storage of the formulation. Therefore, it is advantageous to store the active components separately and only combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example, one or more actives may require a titration period to gradually introduce a patient to a “maintenance” dose. A further example would be if one active requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or user.

Additional problems arise where a multi-drug compound therapy is required, because many users cannot cope with having to use more than one drug delivery system or make the necessary accurate calculation of the required dose combination. This is especially true for users with dexterity or computational difficulties.

Accordingly, there exists a strong need to provide devices and methods for the delivery of two or more medicaments in a single injection or delivery step that is simple for the user to perform.

The disclosed method and system overcomes the above-mentioned problems by providing separate storage containers for two or more active drug agents that are then only combined and/or delivered to the patient during a single delivery procedure. Setting a dose of one medicament automatically controls (e.g., limits) the dose of the second medicament that a user can set. The disclosed method and system also gives the opportunity for varying the quantity of one or both medicaments. For example, one fluid quantity can be varied by changing the properties of the injection device (e.g. dialing a user variable dose or changing the device's “fixed” dose). The settable amount of the second fluid quantity can be changed by varying the properties of the secondary fixed dose mechanism. The disclosed system and method may therefore achieve a wide variety of target therapeutic profiles.

These and other advantages will become evident from the following more detailed description of the invention.

### SUMMARY

The disclosed system and method allows complex combination of multiple drug compounds within a single device. In particular, the disclosed system and method allows the user to set and dispense a multi-drug compound device through a first and second dose setting mechanism and a single dispense interface. A dose limiting system may control the amount of second medicament a user can set using the second dose setting mechanism based on an amount of first medicament the user sets using the first dose setting mechanism. After setting of the first and second medicaments, the first and second medicaments may then be dispensed through the single dispense interface. Although principally described in this application as an injection device, the basic principle could be applicable to other forms of drug delivery, such as, but not limited to, inhalation, nasal, ophthalmic, oral, topical, and like devices.

By defining/controlling the therapeutic relationship between the individual drug compounds, Applicants' delivery device would help ensure that a patient/user receives the optimum therapeutic combination dose from a multi-drug compound device without the inherent risks associated with multiple entirely separate inputs, where the user has to calculate and set the correct dose combination every time they use the device. The medicaments can be fluids, defined herein as liquids, gases or powders that are capable of flowing and that change shape at a steady rate when acted upon by a force tending to change its shape. Alternatively, one of the medicaments may be a solid that is carried, solubilized or otherwise dispensed with another fluid medicament.

This disclosed system is of particular benefit to users with dexterity or computational difficulties as the first variable input and second controlled/limited input (and the associated controlled therapeutic profile) removes the need for them to calculate their prescribed dose every time they use the device and this arrangement allows considerably easier setting and dispensing of the combined compounds.

In an embodiment of the proposed system, a master drug compound, such as insulin, is contained within a primary reservoir and a secondary medicament is contained within a secondary reservoir. Although Applicants' present patent application specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with Applicants' proposed system and method.

For the purposes of Applicants' system and method the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

One embodiment of Applicants' disclosure relates to a drug delivery system to deliver two or more medicaments through a single dispense interface, where the device has a housing containing a first user-operable dose setter operably connected to a primary reservoir of a first medicament containing multiple doses of at least one drug agent. The device also contains a second dose setting mechanism operably connected to a second reservoir of a second medicament containing multiple doses of at least one drug agent. A dose button is operably connected to the primary reservoir of medicament and a single dispense interface is configured for fluid communication with the primary reservoir. The secondary reservoir of a second medicament containing multiple doses of at least one drug agent is configured for fluid communication to the single dispense interface.

This dose button can be any type of mechanism that triggers the delivery procedure, whether driven mechanically or through a combination of electronics and mechanics. The button can move or be a touch sensitive virtual button, for example, a touch sensitive screen. Applicants' system has a single dispense interface configured for fluid communication with the primary reservoir and with a secondary reservoir of medicament containing at least one drug agent. The drug dispense interface can be any type of outlet that allows the two or more medicaments to exit the system and be delivered to the patient. Types of interfaces include hollow needles, catheters, atomizers, pneumatic injectors, or needle-less injectors, mouthpieces, nasal-applicators and the like interfaces.

The secondary reservoir contains multiple doses of medicament. The system is designed such that a single activation of the dose button causes the user set dose of medicament from the primary reservoir and the limited/controlled set dose of medicament from the second reservoir to be expelled through the single dispense interface. By user settable dose it is meant that the user (patient or health care provider) can physically manipulate the device to set a desired dose. Additionally, the user settable dose can be set remotely through the use of wireless communication (Bluetooth, WiFi, satellite, etc.) or the dose could be set by another integrated device, such as a blood glucose monitor after performing a therapeutic treatment algorithm. By limited/controlled set dose it is meant that the user (or any other input) can set or select a dose of medicament from the secondary reservoir, but the amount that is settable is limited or controlled based on the amount of the user settable dose a user sets.

In an example of Applicants' proposed system, a drug delivery device includes a first dose setting mechanism operably coupled to a primary reservoir holding a first medicament. The first dose setting mechanism includes a first dose setter and is a variable dose setting mechanism. The drug delivery device also includes a second dose setting mechanism operably coupled to a secondary reservoir holding a second medicament. The second dose setting mechanism includes a second dose setter. Further, the drug delivery device includes a dose limiting system, wherein the dose limiting system operably couples the variable dose setting mechanism and the fixed dose setting mechanism. Additionally, the dose limiting system is configured to limit a settable amount of a dose of the second medicament a user can set using the second dose setter based on an amount of a variable dose that is set using the first variable dose setter. In an alternative embodiment the first dose setter may be any kind of dose setter, e.g. the first dose setter may be a fix dose setter or an adjustable fix dose setter.

Applicants' present disclosure also covers a housing that includes a body and a linkage mechanism. An inside of the body comprises a first body section and a second body section, wherein the first body section is configured for securely retaining a first drug delivery device and wherein the second body portion is configured for securely retaining a second drug delivery device. In addition, the linkage mechanism operably links the first drug delivery device to the second drug delivery device.

Applicants' present disclosure also covers a method of dispensing a fixed dose of one medicament and a variable dose of a second medicament from separate reservoirs that involves the steps of first setting a dose of a first medicament contained in a primary reservoir of a drug delivery device having a first dose setter. Next, a user may activate a second dose setter to set a dose of the second medicament. The amount of the second medicament that may be set may depend on the amount of the first medicament the user set. Next a dose button is activated that moves both the set dose of the first medicament from the primary reservoir and the limited or controlled dose from the secondary reservoir through a single dispense interface.

The combination of compounds as discrete units or as a mixed unit can be delivered to the body via an integral needle. This would provide a combination drug injection system that, from a user's perspective, would be achieved in a manner that very closely matches the currently available injection devices that use standard needles. One possible delivery procedure would involve the following steps:

1. Attach a single dispense interface, such as a needle hub, to the distal end of the injection device such that the proximal end of the single dispense interface is in fluidic communication with both the primary compound and secondary compound.

2. Dial up (i.e., set) the injection device such that it is ready to dispense the desired dose of the primary compound.

3. Set the injection device such that it is ready to dispense a controlled/limited dose of the second medicament.

4. Insert or apply the distal end of the single dispense interface to the patient at or into the desired administration site. Dose the primary compound by activating a single dose button, which also causes the secondary compound to automatically dispense.

The drug delivery system of Applicants' disclosure may be designed in such a way as to limit its use to exclusive primary and secondary reservoirs through employment of dedicated or coded features.

A particular benefit of Applicants' proposed system and method is that the use of two multi-dose reservoirs makes it is possible to tailor dose regimes when required, especially where a titration period is necessary for a particular drug. In an example, a set of drug delivery devices may be provided that have second dose setting mechanisms and/or reservoirs that have different properties, and thus result in a different fixed dose of a second medicament. The drug delivery devices could be supplied in a number of titration levels with obvious differentiation features such as, but not limited to, aesthetic design of features or graphics, numbering etc, so that a user could be instructed to use the supplied drug delivery devices in a specific order to facilitate titration. Alternatively, the prescribing physician may provide the patient with a number of “level one” titration drug delivery devices and then when these were finished, the physician could then prescribe the next level.

In an example of Applicants' proposed system and method, the drug delivery device is used more than once and therefore is multi-use. Such a device may or may not have a replaceable reservoir of the primary drug compound, but Applicants' disclosed method and system is equally applicable to both scenarios. It is possible to have a suite of different secondary reservoirs for various conditions that could be prescribed as one-off extra medication to patients already using a standard drug delivery device. Should the user attempt to reuse an empty secondary reservoir, Applicants' system could include features that could alert the user to this situation.

A further feature of an example of Applicants' proposed system and method is that both medicaments are delivered via one injection needle and in one injection step. This offers a convenient benefit to the user in terms of reduced user steps compared to administering two separate injections. This convenience benefit may also result in improved compliance with the prescribed therapy, particularly for users who find injections unpleasant, or who have dexterity or computational difficulties. The use of one injection instead of two reduces the possibility for user errors and so may increase patient safety.

As mentioned, in the broadest scope these medicaments could be delivered via a number of routes of administration, for example needle based injections (as described), needle-less injection, inhalation etc. For example, an inhaler version of Applicants' system could have the secondary reservoir containing a liquid, solid or gas form of the second medicament that connects to an MDI or DPI inhaler. The mouthpiece would be part of the single dispense interface. The user would inhale through the mouthpiece, actuating the MDI or DPI inhaler as normal. As the air and medicament passes through the secondary reservoir the second medicament would become entrained in the airflow and delivered to the patient.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates an example drug delivery system, the drug delivery system having two multi-dose reservoirs positioned side-by-side containing a primary medicament and a secondary medicament, respectively;

FIG. 2 illustrates a drug delivery system in accordance with an example of Applicants' disclosure;

FIG. 3a-c illustrate the drug delivery device of FIG. 2 at various phases of the operation of the device;

FIG. 4 illustrates an example possible dose profile achievable with the drug delivery system illustrated in FIG. 2;

FIG. 5 illustrates an example housing for a drug delivery system, in accordance with an example of Applicants' disclosure; and

FIGS. 6a-c illustrate additional example possible dose profiles for drug delivery devices that can be housed in the housing of FIG. 5.

### DETAILED DESCRIPTION

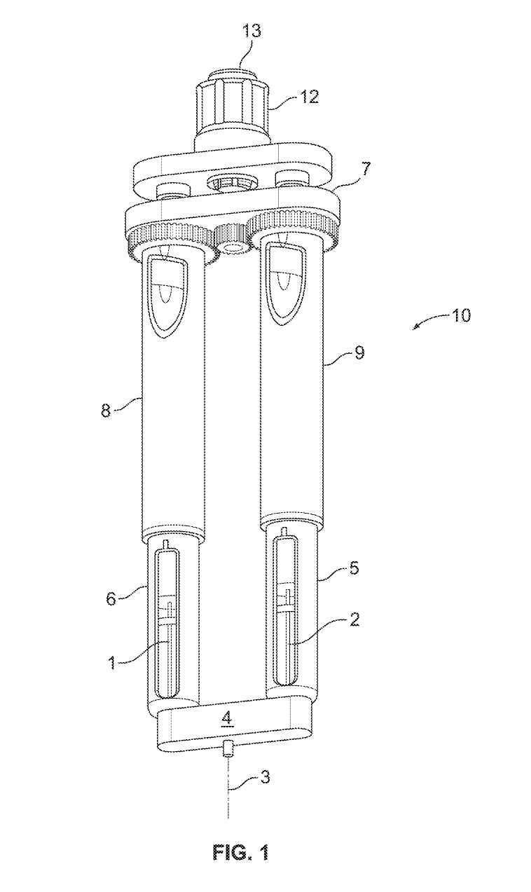

The drug delivery system of the present disclosure administers a variable dose of a first medicament (primary drug compound) and a limited/controlled dose of a second medicament (secondary drug compound) through a single output or drug dispense interface. Setting the dose of the primary medicament by the user automatically controls/limits the settable dose of the second medicament. In an example the drug dispense interface is a needle cannula (hollow needle). FIG. 1 generally illustrates a multi-dose injection device that is capable of setting and delivering both a dose of a first medicament and a second medicament via a single dose setter and a single dispense interface. Such an injection device may be modified such that it is capable of allowing a user to set the dose of the second medicament, where the settable dose of the second medicament is controlled based on the amount of the set dose of the first medicament.

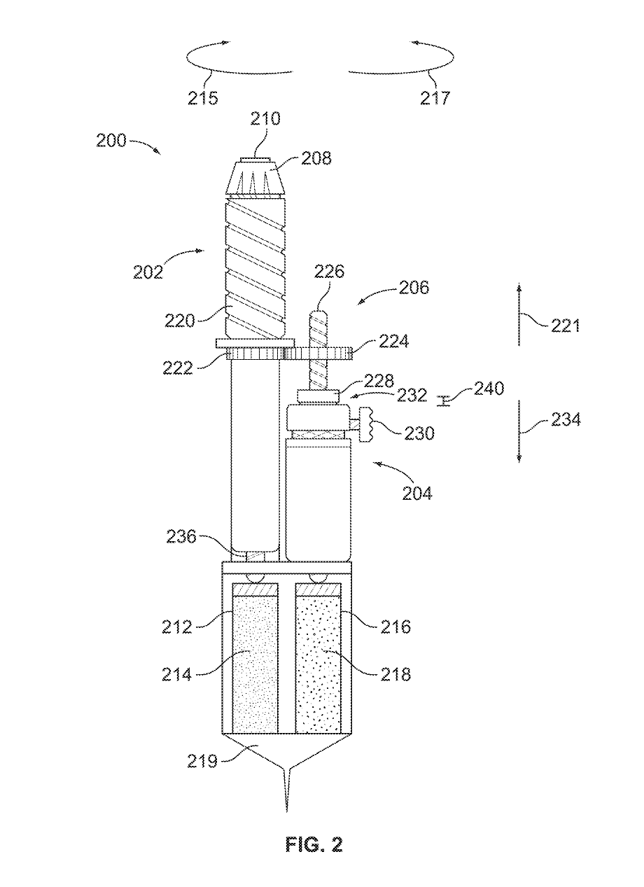

FIG. 2 illustrates an example of such a modified drug delivery device.

In particular, FIG. 1 illustrates one possible example drug delivery system, where a multi-use injection device 10 has two reservoirs that are positioned side-by-side with one containing a first medicament 1 and the other a second medicament 2. These reservoirs may contain multiple doses of each medicament. Each reservoir may be self-contained and provided as sealed and sterile cartridges. These cartridges can be of different volumes and replaceable when empty or they can be fixed (non-removable) in the system. They can also have pierceable seals or septa to accept needle cannula.

The cartridges may be housed in cartridge holders 5 and 6 that have attachment means compatible with a removable, disposable hub or housing 4 that contains the single dispense interface. In this example the single dispense interface is shown as output needle 3. The hub can be of any design, provided that it allows for fluid communication between the primary and secondary medicaments and the single dispense interface or needle 3. An example design of hub 4 would include what is referred to in the art as a “2-to-1 needle” configuration. Although not shown, hub 4 could be supplied by a manufacturer contained in a protective and sterile capsule or container where the user would peel or rip open a seal or the container itself to gain access to the sterile single dispense interface. In some instances it might be desirable to provide two or more seals for each end of the hub. The seal may allow display of information required by regulatory labeling requirements. When a needle is used to deliver the medicaments it is preferred that the hub is designed to be economical and safe for allowing the user to attach a new hub for each injection. Attachment of hub 4 to the multi-use device 10 creates a fluid connection between output needle 3 and medicaments 1 and 2.

The embodiment in FIG. 1 uses a rotational coupling 7 to mechanically link two dose delivery assemblies 8 and 9 in such a way that rotation of single dose setter 12 allows the user to select a dose of the primary medicament 1 and automatically set a fixed or predetermined non-user settable dose of secondary medicament 2. In the embodiment illustrated, the rotational coupling 7 has been embodied as a gear train in which counter-clockwise rotation of the single dose setter causes clockwise rotation of dose dial components (not shown) within the dose delivery assemblies 8 and 9. Rotational coupling 7 may be constructed such that it moves vertically at the same rate as both of the dial components. This allows it to set and dispense both drug compounds throughout the full operational range of the device.

As well understood by those skilled in the art, it is convenient to use lead screws or spindles to push on a piston or bung contained within a cartridge of medicament. As such, it is preferred to use spindles in each dose delivery assembly. By varying the spindle pitches it is possible to vary the dose sizes (and dose ratio) in relation to each other. Specifically, this allows variation of the therapeutic profile to suit a specific therapy or patient requirements by providing devices with different dose ratios. The device shown in FIG. 1 could be operated as follows:

a. Counter-clockwise rotation of the dose setter 12 causes counter-clockwise rotation of the drive gear and clockwise rotation of both driven gears in rotational coupling 7. Clockwise rotation of both driven gears forces both dial components in dose delivery assemblies 8 and 9 to rotate in the same direction and follow a helical path out of the body of the device. This operation allows the user to set a target dose of medicament 1, but not medicament 2, which is automatically set by whatever dose is selected for medicament 1.

b. Initiation of the dosing phase begins with the actuation of dispense or dose button 13 by the user. This causes the dial components to rotate independently of the dose setter.

c. During the dosing phase, the direction of rotation of the single dose setter as well the internal components of both device mechanisms is reversed. The rotational coupling 7 moves back towards the body of the device as both dial components wind back into the mechanisms following their respective helical paths. This reversal of rotation of both mechanisms coupled with the internal overhauling of the spindles by internal drive sleeves (not shown) causes both medicaments to be dispensed in a simultaneous fashion following the fixed ratio profile defined when the user set the target dose of medicament 1.

In addition to altering the relationship of the fixed ratio of medicaments by varying the threaded arrangement of the dose dial components, varying the spindle pitches of the individual device mechanisms in relation to each other may alter the relationship of the fixed ratio of medicaments. Variation of the spindle pitch changes the advance of the spindle during dispense for a given amount of rotation during setting. Differing amounts of advance between the two mechanisms has the effect of creating different dispense ratios between the mechanisms. Variation of the spindle pitches may have the effect of extending the operational window of delivery device 10 in terms of the range of fixed ratios that can be achieved. This may also assist in keeping the spindle pitch in a range that allows resetting should the device be required to be reusable. This means that multiple pen injectors each having a different therapeutic profile can be manufactured. Specifically, this allows variation of the therapeutic profile to suit a specific titration regime and ultimately individual patient requirements.

The attachment means between hub 4 and cartridge holders 5 and 6 can be any known to those skilled in the art, including threads, snap locks, snap fits, luer locks, bayonet, snap rings, keyed slots, and combinations of such connections. The connection or attachment between the hub and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary reservoir to a non-matching injection device.

The shape of the dispense device 10, including hub 4, may be generally oval and/or cylindrical or any other geometric shape suitable for hand manipulation by a user. Additionally, hub 4 could incorporate a safety shield device that would prevent accidental needle sticks and reduce the anxiety experienced by users who suffer from needle phobia. The exact design of the safety shield is not critical to Applicants' drug delivery device, however, an example design is one that is operably connected to the first and/or second reservoirs. In such a design the activation of the safety shield could unlock the drug delivery system or instigate fluid communication between the reservoirs and in some cases cause the second medicament to be dispensed prior to activating the dose button to dispense the primary medicament from the first reservoir. Another example design would physically prevent insertion of the used drug dispense interface into the patient (e.g. a single use needle-guard type arrangement).

As mentioned an example design of Applicants' drug delivery device would include cartridges to contain the medicaments. Cartridges are typically cylindrical in shape and are usually manufactured in glass, sealed at one end with a rubber bung (piston) and at the other end by a rubber septum using a metal ferrule. The dose delivery assemblies are typically powered by a manual action of the user, however, the injection mechanism may also be powered by other means such as a spring, compressed gas or electrical energy.

As mentioned above, a multi-injection drug delivery device such as device 10 may be modified such that it is capable of allowing a user to set the dose of the second medicament, where the settable dose of the second medicament is controlled based on the amount of the set dose of the first medicament. Such a drug delivery device in accordance with an embodiment of Applicants' disclosure is shown in FIG. 2. As seen in FIG. 2, drug delivery system 200 includes a first spindle-type variable dose setting mechanism 202 linked to a second spindle-type dose setting mechanism 204. The devices are linked together via a dose limiting system 206. The dose limiting system 206 acts as a mechanical coupling to operably couple the variable dose setting mechanism 202 and the fixed dose setting mechanism 204. The variable dose setting mechanism 202 is operably coupled to a primary reservoir 212 holding a first medicament 214, and the second dose setting mechanism 204 is operably coupled to a secondary reservoir 216 holding a second medicament 218. Further, the variable dose setting mechanism 202 includes a first dose setter, such as dose dial 208, and the second dose setting mechanism 204 includes a second dose setter, such as dose button 230. The drug delivery device 200 also may include a single dispense interface 219. In this example, the single dispense interface 219 is a ‘two into one needle’; however, in another example, two standard needles could also be used.

The first dose setter 208 may be activated by a user to set a dose of the first medicament 214 and the second dose setter 230 may be activated by the user to set a dose of the second medicament. In the example shown in FIG. 2, the first dose dial 208 may be rotated by a user to set a dose of the first medicament. Further, the dose button 230 may be moved axially by the user to set a dose of the second medicament. However, the axial travel of the button 230 (and therefore level of the second dose that may be set) is limited/controlled by the dose limiting system 206.

In particular, the dose limiting system 206 is configured to control/limit a settable amount of a dose of the second medicament 218 a user can set using the second dose setter 230 based on an amount of a variable dose that is set using the first dose setter 208. The second dose setter 230 is configured to move axially to set a dose of the second medicament 218. In order to limit a settable amount of a dose of the second medicament 218 a user can set using the second dose setter 230, the dose limiting system 206 limits a maximum axial distance the second dose setter can travel based on the amount of the variable dose that is set using the first dose setter 208.

The variable dose setting mechanism 202 may include a dial sleeve that is coupled to the dose limiting system 206, such as dial sleeve 220. The dose limiting system 206 includes a drive gear 222 and a driven gear 224 coupled to one another. The driven gear 224 has an internal thread (not shown). The dose limiting system also includes a spindle 226 that is threadedly engaged to the internal thread of the driven gear. Further, the dose limiting system 206 includes a stopper 228 that is threadedly engaged to the spindle 226. In combination with each other, these elements of the dose limiting system 206 facilitate limiting the settable dose of the second medicament based on the amount of the first medicament set by a user.

During dose setting, activation of the first dose setter 208 rotates the drive gear 222 in a first rotational direction 215. The drive gear 222 is coupled to the dial sleeve 220 in such a way that rotation of the dial sleeve 220 causes rotation of the drive gear 222. This rotation of the drive gear 222 in turn rotates the driven gear 224 in a second rotational direction 217 opposite the first rotational direction. While both the drive gear 222 and the driven gear 224 are allowed to rotate, they are constrained axially. The dose setting mechanism may be capable of transmitting torque to the drive gear throughout its full range of axial travel (i.e., as it moves further out of the body). In an example, the first rotational direction 215 is clockwise and the second rotational direction is counterclockwise. Rotation of the driven gear 224 forces the spindle 226 to travel axially in proximal direction 221 through the internal thread of the driven gear 224. The axial travel of the spindle 226 lifts the stopper 228 a corresponding amount in direction 221, and this increases a gap length 240 of the gap 232 between the stopper 228 and the second dose setter 230. The increasing gap 232 permits the second dose setter to move further axially towards the stopper potentially setting the second medicament 218.

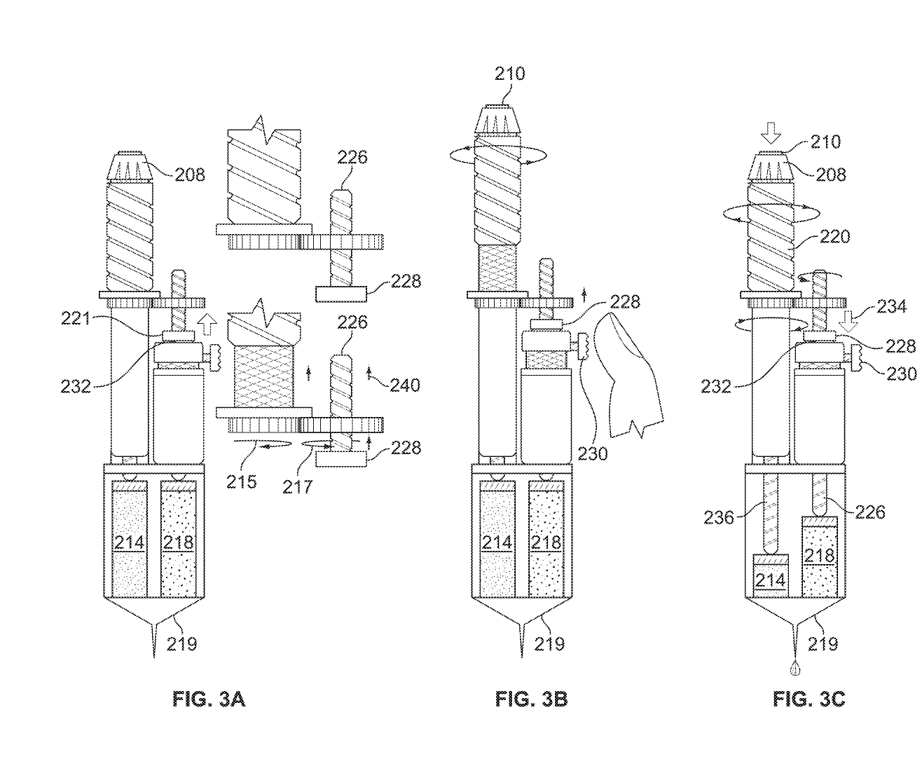

In this example, the first, variable dose setting mechanism 202 is a rotate-to-set-and-dispense mechanism that follows a helical path out of (and back into) the housing of the device. Such rotate-to-set-and-dispense mechanisms are well-known in the art. The operation of the drug delivery device 200 includes the following general phases: (i) setting of the first medicament 214 and the second medicament 218, (ii) initiation of dispense, (iii) dispense of the first medicament 214, and (iv) dispense of the second medicament 218 along with the remainder of the first medicament 214. These steps or phases are described in greater detail below with reference to FIGS. 3a-c.

During dose setting (which is depicted in FIGS. 3a-b), rotation of the dose setter 208 sets the variable dose of the first medicament 216. This rotation of the dose setter 208 (through the mechanical coupling of the dose limiting system 206) forces the spindle 226 with integrated stopper 228 to climb the internal thread on the inside of the driven gear 224. As the spindle 226 climbs the thread of the driven gear 224 the length 240 of the gap 232 between the top of the button 230 and the stopper 228 increases.

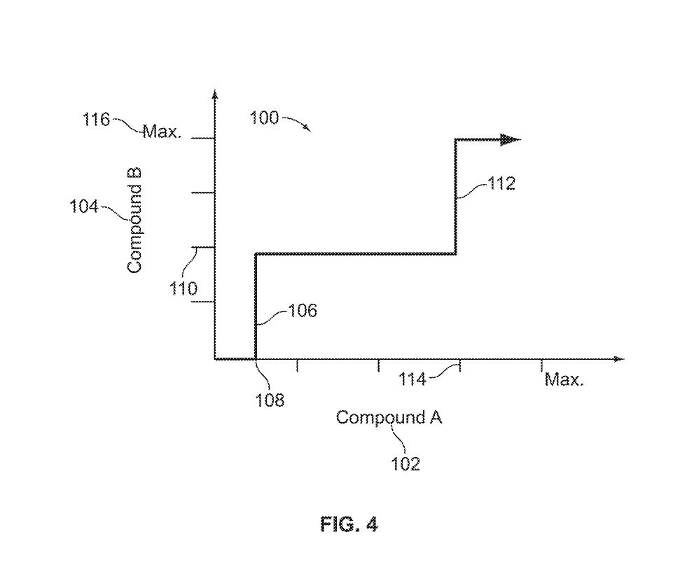

The dose volumes of the drugs are set in accordance with a stepped fixed dose-variable dose relationship between the two medicaments (such as profile 100 shown in FIG. 4). A predetermined fixed amount of axial lift of the stopper 228 (i.e., a definite increase in the gap length 240 between the button 230 and the stopper 228) allows the user to set the fixed dose (for example the maximum permissible fixed dose or a predefined fraction of this maximum dose) by controlling how far the user can pull out the dose mechanism button 230. In this example, the fixed dose mechanism is set manually by the user using the dose button 230. Should this gap 232 not reach the predefined threshold, the user can attempt to set the fixed dose mechanism 204 but will be prevented from completing the dose set due to the restricted movement provided by the stopper 228.

In an example, the user must set a minimum amount of the first medicament 214 before the second dose mechanism reaches a point where it can be set. Below this threshold the second dose mechanism cannot be set, however, the first dose setting mechanism 202 may be set and can dispense the first medicament.

Ratcheted intervals in the fixed dose mechanism 204 may help achieve the minimum settable fixed dose and the stepped fixed dose relationship. As such, the fixed dose mechanism may comprise a number of ratchet intervals. Each interval may define a full dose or fixed part thereof. As such, the ‘dose’ is either set or not depending on whether the mechanism has reached the next ratchet interval. The amount that the mechanism can move is restricted by the dose limiting system. In an example, (i) should the distance required to reach a ratchet interval (fixed dose or fixed part thereof) be, for example, 5 millimeters (mm) of travel, and (ii) the dose limiting system restricts the travel of the fixed dose mechanism to 3 mm, then the fixed dose mechanism 204 would be unable to set that dose. In this example, until the dose limiting system allows 5 mm travel, then no fixed dose could be set and delivered. However, should the dose limiting system permit more than 5 mm of travel (e.g., 5.1 mm of travel), then the first step in the profile is achieved as the fixed dose can be set and dispensed. Should the fixed dose mechanism permit setting fixed fractions defined by ratchet intervals, and should the dose limiting system permit say 15.1 mm travel (defined by the variable dose set by the user), then the fixed dose mechanism could reach the third ratchet (i.e., the third effective step on the profile).

After setting the dose of the first medicament and the fixed dose of the second medicament (assuming that the threshold to allow setting of the fixed dose has been met), the user may initiate the dispense process. The dispense process is depicted in FIG. 3c. The user may initiate the dispense process by actuating the dose button 210. Actuation of the dose injection button 210 allows a drive sleeve (not shown) to move axially back into the device and for the dial sleeve 220 to rotate back into the device. As mentioned above, such a dose setting mechanism having a drive sleeve and dial sleeve that operate in this way is well known in the art. Rotation of the drive sleeve rotates the drive gear 222 and hence the driven gear 224 in the opposite direction. This action moves the spindle 226 and stopper 228 downward in axial direction 234 towards the button 230 of the second dose setting mechanism 204.

During this phase the drive sleeve moves axially back into the device causing the first medicament 214 to be dispensed through the single dispense interface 219. In particular, the spindle 236 of the variable dose component 202 is overhauled by the drive sleeve and is forced to advance thus dispensing the first medicament 214. The dial sleeve 220 rotates in the opposite direction (back into the housing of the device) from dialing during the dispense phase. The second medicament 218 is not dispensed until the spindle 226 and stopper 228 have move sufficiently downwards to close any gap 232 between the stopper 228 and the button 230 of the fixed dose component 204. However, at a given point during dispense, the stopper 228 comes into contact with the button 230. As mentioned above with respect to dispense of the variable dose setting mechanism 202, rotation of the dial sleeve 220 back into the housing of the device also rotates the drive gear 222 and hence the driven gear 224 in opposite directions. This forces the spindle 226 and stopper 228 to move further downwards against the button 230, and this causes the second medicament 218 to be dispensed (assuming that the fixed dose setting threshold has been met). If the threshold has not been met and the button 230 is partially out, the stopper 228 will push it back in but no dose will have been dispensed. If the threshold has been met, the stopper 228 will engage with the fixed dose mechanism 204 and dispense the dose. However, if the threshold has not been met and the fixed dose mechanism 204 has not been set (i.e., button not moved out) the stopper 228 will simply return to the start position and only just engage with the fixed dose mechanism 204 at the end (i.e., the original start position).

In an example of Applicants' proposed concept, the settable dose of the second medicament could be divided into smaller doses such that in instances where a low amount of variable first medicament is required, a proportionally lower amount of the second medicament could be set. In this instance, the stopper would travel a smaller amount; however, this would still allow the user to raise the button to a proportionally smaller set position.

The threshold points to which the second dose setting mechanism 204 can be set may be altered through the variation of the parameters of the mechanical coupling and dose limiting system 206. For example, varying the relative diameters between the drive gear and driven gear may alter the settable dose volume of the second medicament. As another example, the same effect may be realized by altering the pitch of the internal threads of the driven gear 224 (and/or on the spindle 226).

As yet another example, the settable dose volume of the second medicament be altered using the ratchet pitch or spindle pitch depending upon the fixed dose mechanism embodiment. This means that multiple pen injectors each having a different therapeutic profile may be manufactured. Specifically, this allows variation of the therapeutic profile to suit a specific titration regime or ultimately individual patient requirements. These two design variables can be used independently to achieve the desired fixed dose set point or in combination. In combination, they may have the effect of extending the operational window of the device in terms of the range of fixed dose set points that can be achieved. This means that multiple pen injectors each having a different therapeutic profile can be manufactured. Specifically, this allows variation of the therapeutic profile to suit a specific therapy or patient requirements.

Drug delivery device 200 may be useful for certain therapies where it is beneficial for the dose of the second medicament to increase in fixed dose stepped increments as the corresponding dose of the first medicament increases. An example profile 100 is shown in FIG. 4. Such a profile is advantageous for certain therapies where it is beneficial for the dose of the second medicament 104 to increase in fixed stepped increments as the corresponding dose of the first medicament 102 increases. Each of these stepped increases only occurs once a specific predefined threshold dose of the first medicament has been exceeded. In this example, the first step 106 occurs when a threshold dose 108 of the first medicament 102 is set. The first step 106 results in a dose 110 of the second medicament 104 being set. The second step 112 occurs when a threshold dose 114 of the first medicament 102 is set. The second step 112 results in a dose 116 (which may, for example, be a maximum dose) of the second medicament 104 being set.

The relative spacing between these threshold values of the first medicament may or may not be regular. Profiles of this type are not achievable from a combination drug that is co-formulated into a single primary pack (such as, but not limited to, a standard 3 ml glass cartridge) where the concentration of each of the various constituent parts is constant (xmg/ml, ymg/ml). A dose limiting profile such as profile 100 is advantageous in reducing the potential for overdose of one of the drugs in cases where two drug compounds are administered in a single injection.

In addition, although shown as a “2-to-1” needle, the injection component could be embodied as two separate needles. A separate needle would be provided for each separate medicament. In addition, the disclosed drug delivery system could be embodied in such a way as to allow for the injection of drug compounds from more than two primary packs. This would involve the addition of additional drive mechanisms and an extension of the dependant linking mechanisms.

The disclosed drug delivery system may be suited towards a modular disposable or re-usable platform in terms of managing drug wastage. This is because there is a risk of one medicament being finished before the other unless there is a strict 1:1 ratio between the two medicaments. However, where each side is resettable, new primary packs can be inserted and the device can continue to be used. Possible embodiments for a modular disposable platform could, but are not limited to, involve the replacement of the entire dose specific device mechanism fitted with a new primary pack. Suitable re-engagement features may be integrated into the device platform to facilitate the alignment and fastening of the individual device mechanisms together in a robust and user friendly fashion. It is possible that such features could be arranged to define the permissible functionality of the two individual elements on their own.

A possible re-usable platform would feature spindles that could be back wound into their respective devices once they had reached the limits of travel. In addition to this functionality, the platform would feature a means of replacing both primary packs after the resetting of one or both spindles.

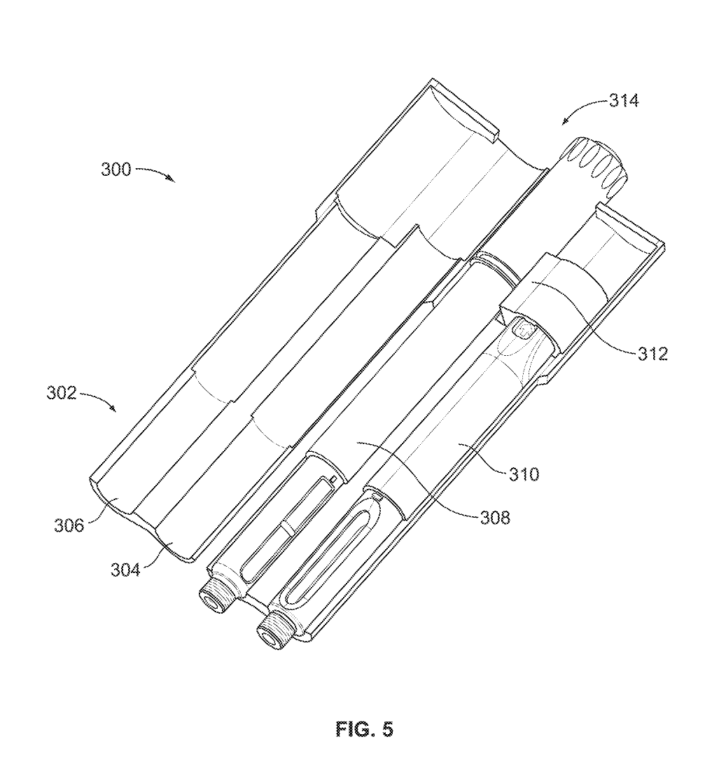

In an example, a drug delivery device such as drug delivery device 200 may be held in a housing such as the housing 300 depicted in FIG. 5. Beneficially, the housing may allow for a link between two drug delivery device components and give the appearance and feel to a user of a single, unitary drug delivery device. The housing 300 includes a main body 302. An inside of the main body comprises a first body section 304 and a second body section 306. The first body section 304 is configured for securely retaining a first drug delivery device 308, and the second body portion 306 is configured for securely retaining a second drug delivery device 310. As can be seen in FIG. 5, each body portion is molded to match the shape of the intended drug delivery device.

The housing 300 also comprises a linkage mechanism 312. The linkage mechanism 312 operably links the first drug delivery device 308 to the second drug delivery device 310. In an example, the linkage mechanism 312 is attachable to (i) a first dose setting mechanism of the first drug delivery device and (ii) a second dose setting mechanism of the second drug delivery device. Many types of linkage mechanism are possible. Generally, the linkage mechanism may be any mechanical connection that operably links the two devices, such that two devices may be used in conjunction with one another to deliver a first medicament and a second medicament. For example, the linkage mechanism and drug delivery devices described above with respect to FIG. 2 are possible. However, other linkage mechanisms and drug delivery devices are possible as well. The drug delivery devices that are intended to be used with the housing may be configured to connect to and operate with the linkage mechanism of the housing.

In an example, the housing 300 may be a modularly reusable housing. When the medicament of one of the inserted drug delivery devices is depleted, the user may open the housing, remove the depleted device, and replace the depleted device with a new device. Similarly, the same process may occur when the other drug delivery device becomes depleted. It should be understood that that devices may become depleted at the same time or at different times. In an example, the body is hinged about an axis 314. A user can open the body to insert and remove the two devices. In order to remove a depleted device, the user may open the hinged housing (e.g. to the position shown in FIG. 5). The user may then remove the appropriate depleted device and insert a new device. It should be appreciated that when the body is closed, the housing gives the appearance and feel of a single, unitary drug delivery device.

In an example, the housing may be a reusable housing for use with reusable drug delivery devices. In this embodiment, when an inserted drug delivery device becomes depleted, a user may remove the drug delivery device. The user may then reset the drug delivery device and inserted a new medicament cartridge. The user may then reinsert the reusable drug delivery device in the housing. In both cases, the advantage is in supplying two devices inter-locked to each other for the purposes of controlled combined delivery.

In other examples, the housing may be a disposable housing (i.e., one that is not intended to be reused). In this example, when one of the inserted drug delivery devices has become depleted, the housing (and housed drug delivery devices) may be disposed of.

Additionally, in an example, there may be interlocks within the linkage mechanism or housing such that the combined device will not function unless there are two devices fixed in position with sufficient medicament remaining to provide the intended therapeutic profile of the device. For instance, the interlocks may be mechanical or electronic such that only when both mechanisms are within the housing can the combination of devices be used. For example, the interlock may be a pin that locks the device mechanism to the housing such that nothing can be set or dispensed until the other device is inserted. The act of inserting the second device or closing the housing with the other device present may release the pin and allow the mechanism to move freely.

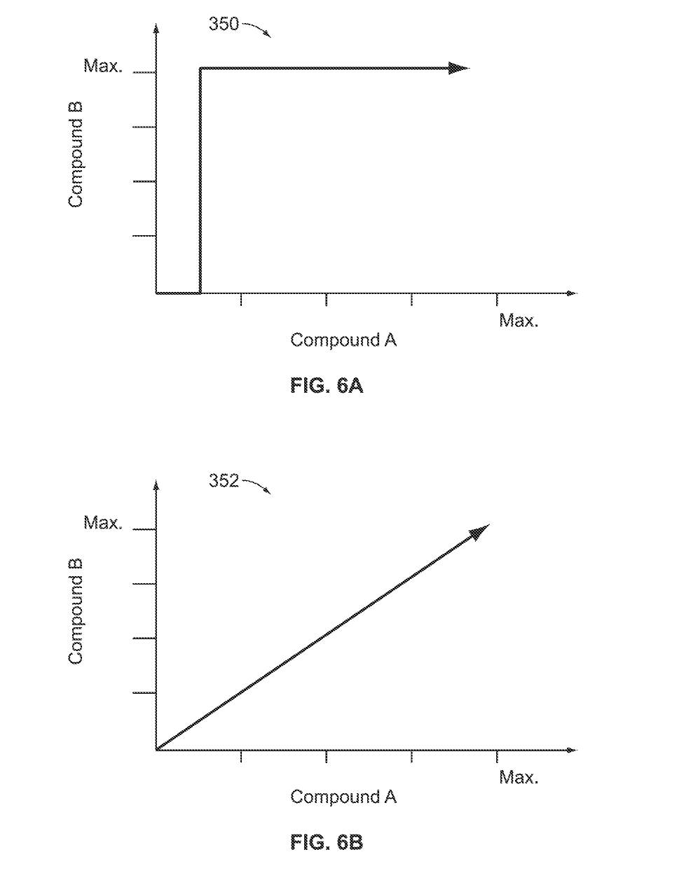

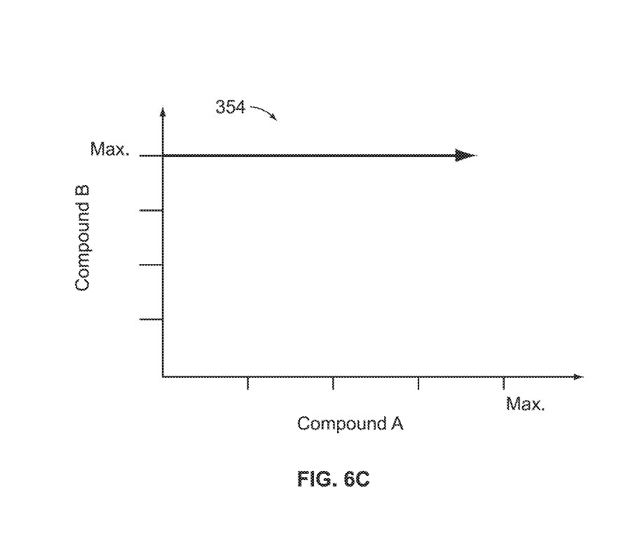

The intended dose profile of a dual-device system that may be used with housing 300 may be any possible therapeutic profile for delivery of two medicaments (e.g., simultaneous, sequential, interspersed delivery of two medicaments). Example dose profiles include, but are not limited to a multilevel fixed dose/variable dose profile (e.g., the profile 100 shown in FIG. 4), a delayed fixed dose therapeutic profile (e.g., profile 350 shown in FIG. 6a), a fixed ratio therapeutic profile (e.g., profile 352 shown in FIG. 6b), or a variable dose (of a first medicament)/fixed dose (of a second medicament) therapeutic profile (e.g., profile 354 shown in FIG. 6c).

Exemplary embodiments of the present invention have been described. Those skilled in the art will understand, however, that changes and modifications may be made to these embodiments without departing from the true scope and spirit of the present invention, which is defined by the claims.

1. A drug delivery device comprising:
a first dose setting mechanism operably coupled to a primary reservoir holding a first medicament, the first dose setting mechanism comprising a first dose setter;
a second dose setting mechanism operably coupled to a secondary reservoir holding a second medicament, the second dose setting mechanism comprising a second dose setter;
a dose limiting system, wherein the dose limiting system operably couples the first dose setting mechanism and the second dose setting mechanism, and wherein the dose limiting system is configured to limit a settable amount of a dose of the second medicament based on an amount of a set dose of the first medicament.

2. The drug delivery device of claim 1, wherein the second dose setter is configured to move axially to set the dose of the second medicament, and wherein the dose limiting system limits a maximum axial distance the second dose setter can travel.

3. The drug delivery device of claim 2, wherein the dose limiting system comprises:
a drive gear;
a driven gear having an internal thread, wherein the driven gear is coupled to the drive gear;
a spindle threadedly engaged to the internal thread of the driven gear; and
a stopper engaged to the spindle,
wherein the second dose setter is axially moveable between a portion of the second dose setting mechanism and the stopper,
wherein, during dose setting, activation of the first dose setter rotates the drive gear in a first rotational direction, wherein rotation of the drive gear rotates the driven gear in a second rotational direction opposite the first rotational direction, wherein the rotation of the driven gear forces the spindle to travel axially through the internal thread of the driven gear, and wherein the axial travel of the spindle lifts the stopper and increases a gap length between the stopper and the second dose setter, thereby permitting the second dose setter to be capable of further axial movement towards the stopper in order to set a dose of the second medicament.

4. The drug delivery device of claim 1, wherein the first dose setting mechanism must be set a minimum amount before the second dose setting mechanism can be used to set a dose of the second medicament.

5. The drug delivery device of claim 1 configured to set a dose of the second medicament once a minimum dose of the first medicament is set.

6. The drug delivery device of claim 1, wherein the first dose setter is a dose dial, and wherein the second dose setter is a button that is axially moveable.

7. The drug delivery device of claim 1, further comprising an outer housing, wherein the outer housing houses the first and second dose setting mechanisms.

8. The drug delivery device of claim 1, wherein the first dose setting mechanism is a variable dose setting mechanism.

9. A housing comprising:
a main body, the main body comprising a first body section and a second body section;
a first drug delivery device retained in the first body section;
a second drug delivery device retained in the second body section; and
a linkage mechanism, the linkage mechanism operably linking the first drug delivery device 308 to the second drug delivery device.
a first dose setting mechanism operably coupled to a primary reservoir holding a first medicament, the first dose setting mechanism comprising a first dose setter;
a second dose setting mechanism operably coupled to a secondary reservoir holding a second medicament, the second dose setting mechanism comprising a second dose setter;
a dose limiting system, wherein the dose limiting system operably couples the first dose setting mechanism and the second dose setting mechanism, and wherein the dose limiting system is configured to limit a settable amount of a dose of the second medicament based on an amount of a set dose of the first medicament.

10. The drug delivery device of claim 9 wherein the linkage mechanism is attachable to the first dose setting mechanism of the first drug delivery device and the second dose setting mechanism of the second drug delivery device.

11. The drug delivery device of claim 9 further comprising an interlock, the interlock allowing the drug delivery device to function only if the first and second device is fixed in position with sufficient medicament remaining to provide an intended therapeutic profile.

12. A housing comprising:
a body, wherein an inside of the body comprises a first body section and a second body section; and
a linkage mechanism,
wherein the first body section is configured for securely retaining a first drug delivery device, wherein the second body portion is configured for securely retaining a second drug delivery device, and wherein the linkage mechanism operably links the first drug delivery device to the second drug delivery device.

13. The housing of claim 12, wherein the linkage mechanism is attachable to (i) a first dose setting mechanism of the first drug delivery device and (ii) a second dose setting mechanism of the second drug delivery device.

14. The housing of claim 12, wherein the housing is modularly reusable

15. The housing of claim 9, wherein the body is hinged
